(19) United States
(12) Patent Application Publication
(10) Pub. No.: US 2026/0212977 A1
(43) Pub. Date: Jul. 23, 2026

(54) Dynamic Operative and Procedural Report Generator

(71) Applicant: Jonathan Katz, Baltimore, MD (US)

(72) Inventor: Jonathan Katz, Baltimore, MD (US)

(21) Appl. No.: 19/032,075

(22) Filed: Jan. 19, 2025

(51) Int. Cl. G16H 15/00

(52) U.S. Cl. CPC G16H 15/00

(57) ABSTRACT

The Dynamic Operative and Procedural Report Generator is a system designed to create structured operative and procedural reports dynamically. The system receives procedure-specific inputs from a user and, based on predefined templates and logic, requests additional information to ensure a complete and accurate report. The invention allows for seamless customization across various medical specialties and integrates with electronic medical records (EMR) systems. The generated reports follow a structured format, ensuring consistency, efficiency, and accuracy in medical documentation. The system can be implemented as a web application, desktop application, or standalone software, with security features including encryption and role-based access. The invention significantly reduces the time required for medical professionals to document procedures while maintaining compliance with medical documentation standards.

### BACKGROUND OF THE INVENTION

Medical professionals are required to produce accurate and detailed documentation for every procedure performed. Traditional methods rely heavily on manual data entry or static templates, which are time-consuming, prone to errors, and lack flexibility. Existing solutions often fail to account for the dynamic nature of medical procedures and the need for tailored documentation based on patient-specific details. This invention addresses these limitations by providing a system that dynamically generates operative and procedural reports based on user input, ensuring accuracy and efficiency.

### BRIEF SUMMARY OF THE INVENTION

The invention is a platform designed to create customized operative and procedural notes dynamically. Users input specific details such as the procedure type, affected body part, anesthesia type, and additional parameters. Based on these inputs, the system dynamically requests further information required to complete the documentation accurately. The system processes these inputs using a backend algorithm that populates pre-defined templates with the relevant data. The final output is a professionally formatted report that can be integrated into EMR systems, stored digitally, or printed for physical records. The interface can be implemented as web-based, desktop-based, or mobile-based, depending on the environment of use.

### BRIEF DESCRIPTION OF THE DRAWINGS

FIG. 1: User Interface Layout—Depicts the input fields for procedure type, affected area selection, anesthesia, and other options.

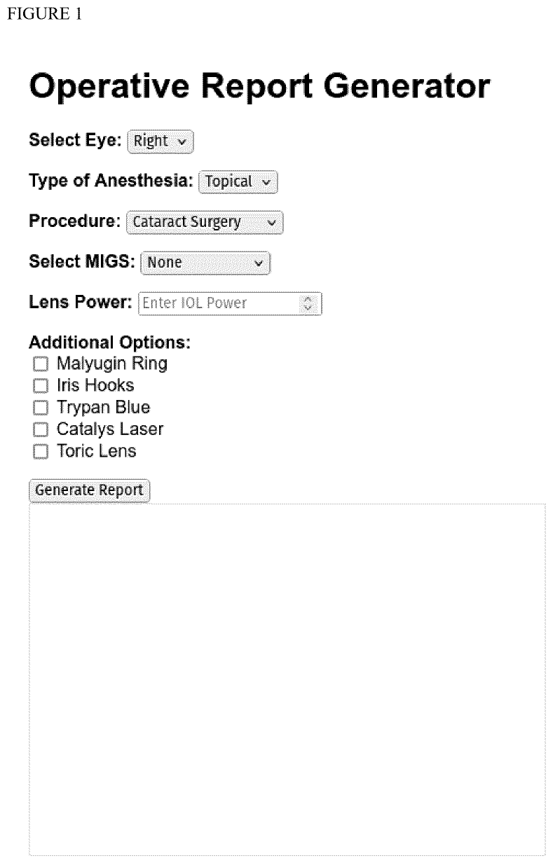

FIG. 2: System Architecture—Illustrates the backend processing workflow from user input to report generation.

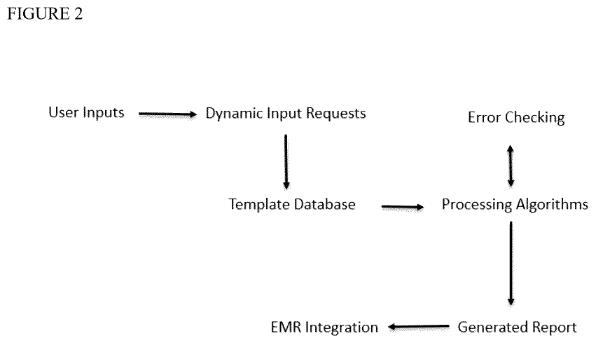

FIG. 3: Sample Generated Report—Shows a completed procedural note based on sample inputs.

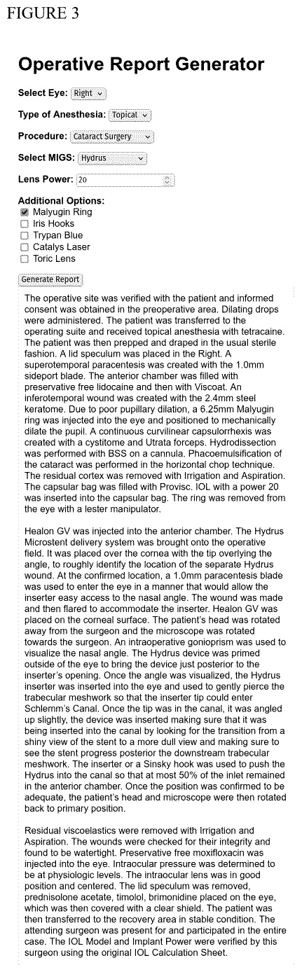

### DETAILED DESCRIPTION OF THE INVENTION

The system comprises the following components:

### 1. User Interface

Input fields for procedure details, patient-specific information, and customization options.
Can be implemented as a web application, desktop application, or mobile application.
Dropdown menus and free-text fields for pre-defined and custom parameters.
Dynamically requests additional inputs based on prior user selections to ensure completeness.

### 2. Backend Processing

A database containing templates for various procedures across multiple medical fields.
Algorithms that dynamically substitute placeholders in templates with user-provided data.
Error-checking mechanisms to ensure consistency and prevent omissions.

### 3. Output

Professionally formatted reports in PDF, Word, or other document formats.
Integration with EMR systems via APIs or standard data exchange protocols.
Options for digital signatures and secure transmission.

### 4. Security Features

Encryption for data transmission and storage.
Role-based access control for different user types (e.g., doctors, assistants).

### 5. Flexibility

Configurable to accommodate the unique documentation needs of various medical specialties, such as cardiology, orthopedics, general surgery, and more.

1. A system for generating customized operative reports, comprising an interface for receiving procedure-specific inputs, a backend processing module that dynamically substitutes user inputs into predefined templates, and a report generation module that outputs formatted reports in digital or print-ready formats.

2. The system of claim 1, further comprising an integration module for exporting reports to electronic medical record systems.

3. The system of claim 1, wherein the interface can be implemented as a web application, desktop application, or mobile application.

4. The system of claim 1, wherein the interface dynamically requests additional inputs based on previously entered information.

5. The system of claim 1, wherein the backend processing module includes an error-checking algorithm to validate user inputs.

6. The system of claim 1, further comprising encryption mechanisms to ensure secure data handling.

7. The system of claim 1, wherein the templates and workflows can be customized for different medical specialties
